(19) United States
(12) Patent Application Publication
(10) Pub. No.: US 2017/0224282 A1
(43) Pub. Date: Aug. 10, 2017

(54) TEMPERATURE SENSING PATCH

(71) Applicant: Chu-Yih Yu, Taipei (TW)

(72) Inventor: Chu-Yih Yu, Taipei (TW)

(21) Appl. No.: 15/015,137

(22) Filed: Feb. 4, 2016

(51) Int. Cl. A61B 5/00; A61B 5/01

(52) U.S. Cl. CPC A61B 5/6833; A61B 5/01; A61B 5/0008; A61B 5/742; A61B 2560/0456; A61B 2562/0271; A61B 2560/0475; A61B 2560/0214; A61B 2562/227

(57) ABSTRACT

A temperature sensing patch that is easy to use and capable of efficiently monitoring temperature change in human, body for a long time, capable of being used with a reading device, comprising the temperature sensing patch configured to attach to human skin, comprising a housing, a circuit board, a temperature sensing unit electrically connected with the circuit board, a battery, a processor chip, at least a memory unit, and at least a port; wherein the processor chip can automatically turn on the temperature sensing patch and reset the previous data by a predetermined program, rather than through a manual switch, when the temperature sensing patch is removed from the reading device.

### FIELD OF THE INVENTION

The present invention is relates to a temperature sensing patch, particularly to a temperature sensing patch capable of being used for medical assistance.

### BACKGROUND OF THE INVENTION

There is no two exactly the same green leaves, and there is no exactly the same gene between people, everyone taking the same medicine may not be effective, everyone's reactions to drugs are different, so in general, drug development from pharmaceutical companies or doctor's prescription, due to the technical limitations, can only treat the patient by “average level”, namely standardized medical care.

However, with the introduction of high throughput sequencing technology and the development of large data processing capabilities, modern medical science is undergoing tremendous change from the traditional standardized medical care model to a personalized medicine paradigm, the personalized medicine that is “precision medicine” has become a new direction in modern medicine.

The purpose of the precision medicine is based on the patient's greatest wellbeing and the effective allocation of spending on socialized medicine, combined with modern epidemiology and preventive medicine, clinical diagnostics, therapeutics, molecular medicine, medical informatics technology, economics of health and medical sociology to let the traditional medicine move towards modernization and provide disease prevention, screening, diagnosis, treatment and rehabilitation program for everyone like tailored according to everyone's figure, with minimal resources to maximize health care, and improve the health of the overall population.

On the other hand, the Traditional Chinese Medicine (TCM) theory holds that the human body is viewed as a whole system that has nature operation and balance, the human will get sick if human body is out of balance, and the “different treatment according to the same sickness nature” already exist in the therapeutic strategy of the TCM that is corresponding to the “precision medicine” theory. That's because everyone's physiques are different, the same treatment will generate different reactions and results, it is necessary for each patient to be diagnosed by pathology and etiology, in order to make the most appropriate treatment.

How can the precise medicine be combined with the TCM, combined the modem technology and traditional medical method with the scientific knowledge of the nature of the human body and disease to achieve the symmetry precision medicine which is major problem.

In order to allow the TCM to achieve the precise medicine effect, it is very important to select the correct symmetric life information, which is a key marker of biology and physics; because the symmetric life information and the information gap [a state of balance] vary from person to person and from disease to disease, the medication and intervention are based o these situations, the so-called symmetric balanced medical care that has direction in personalized medicine.

So, to select symmetric temperature change as a key marker is a preferable choice, which has five following reasons, 1^{th}: humans are endotherms; 2^{nd}: temperature is a statistics expression for the average kinetic energy of molecule; 3^{rd}: it is a greatest common divisor for a variety of medicine; 4^{th}: it is the most accurate, convenient, and cheapest way of life measuring way; 5^{th}: it is symmetry and symmetry breaking theory based on physics frontier in the 21st century.

Although it has been approved that the symmetric temperature change can help to realize the symmetry precision medicine. Currently, the biggest problem is there is no any device that can be used to obtain a symmetric temperature change.

In view of the above, how to effectively obtain a symmetric temperature change data and apply in the measurement of body temperature of the precision medicine has become the subject of the present invention intended to be improved.

### SUMMARY OF INVENTION

The present invention is to provide a temperature sensing patch capable of monitoring and recording the body temperature change.

For solving the above-mentioned problem and achieving the purpose of the present invention, the technical scheme discloses a temperature sensing patch capable of being used with a reading device (A), comprising the temperature sensing patch (100) capable of being attach to human skin, comprising a housing (1), a circuit board (2), a temperature sensing unit (3) electrically connected with, the circuit board (2), a battery (4), a processor chip (5), at least a memory unit (6), and at least a port (7); wherein the processor chip (5) can automatically turn on the temperature sensing patch (100) and reset the previous data by a predetermined program, rather than through a manual switch, when the temperature sensing patch (100) is removed from the reading device (A).

More preferably, wherein the housing (1) comprises a receiving space (11) defined therein; wherein the circuit board (2) is located within the received space (11); wherein the temperature sensing unit (3) electrically connected with the circuit board (2) defines a sensing terminal (31) with one end thereof protruded over a surface of the housing (1) to contact with the human skin for sensing temperature; wherein the battery (4) is installed within the receiving space (11); wherein the processor chip (5) is installed on the circuit board (2) and be capable of dealing with the continuous digital data transmitted from the temperature sensing unit (3) within a predetermined time; wherein the, memory unit (6) is installed on the circuit board (2) and stored the continuous digital data transmitted from the processor chip (5); and wherein the port (7) is installed within the receiving space (11), one end of which is located at a peripheral edge of the housing) for connecting with the reading device.

More preferably, wherein the temperature sensing patch (100) further comprises a light unit (8) electrically connected with the circuit board (2) and located within the received space (11) for a light indication of operation status.

More preferably, wherein the housing (1) comprises a through hole (12) corresponding to the sensing terminal (31).

More preferably, wherein the temperature sensing patch (100) further comprises an adhesive unit (9) disposed on a surface of the housing (1) and located around a periphery of the through hole (12) for attaching on the human skin.

More preferably, wherein the adhesive unit (9) comprises an adhesive, layer (91) and a release layer (92) laminating with the adhesive layer (91).

More preferably, wherein the housing (1) further comprises a first housing (13) and a second housing (14) correspondingly disposed on the first housing (13).

More preferably, wherein the second housing (14) comprises, a receiving groove (141) disposed in a side on top thereof and an identification sticker (142) disposed in the receiving groove (141) and configured to identify the temperature sensing patch (2).

More preferably, wherein the battery (4) capacity must be at least 24 hours for the temperature sensing patch (100) operation.

More preferably, wherein the port (7) is one of the following: Type-A USB port, Type-B USB port, Type-C USB port, Micro-A USB, Micro-B USB, Mini-A USB port, Mini-B USB port.

More preferably, wherein the light unit (8) is a LED light.

Compared to prior art, the advantages of the present invention are: When in use, the temperature sensing patch can be used by multi-point monitoring method to monitor the temperature change of the human body for a long period time, so the temperature sensing patch is not as familiar as traditional thermometer does, it can provide the more practical reference data to the precision medicine. Besides, there is no power switch in the temperature sensing patch, so that it can automatically turn on the power through the predetermined program when the temperature sensing patch is removed from the reading device, and then the temperature sensing patch will turn itself off when the battery runs out of power. The design can reduce user's burden on operation.

### BRIEF DESCRIPTION OF THE DRAWINGS

FIG. 1 is a schematic view of the present invention;

FIG. 2 is an exploded view of the present invention;

FIG. 3 is the schematic from another angle;

FIG. 4 is the exploded view from another angle;

FIG. 5 is a flow view showing temperature sensing patches applied in a patient; and

FIG. 6 is a schematic view showing the temperature sensing patches being removed from the patient and plugged into a reading device to obtain the temperature data.

### DETAILED DESCRIPTION OF THE PREFERRED EMBODIMENTS

The following description is a preferable embodiment according to the Figs.

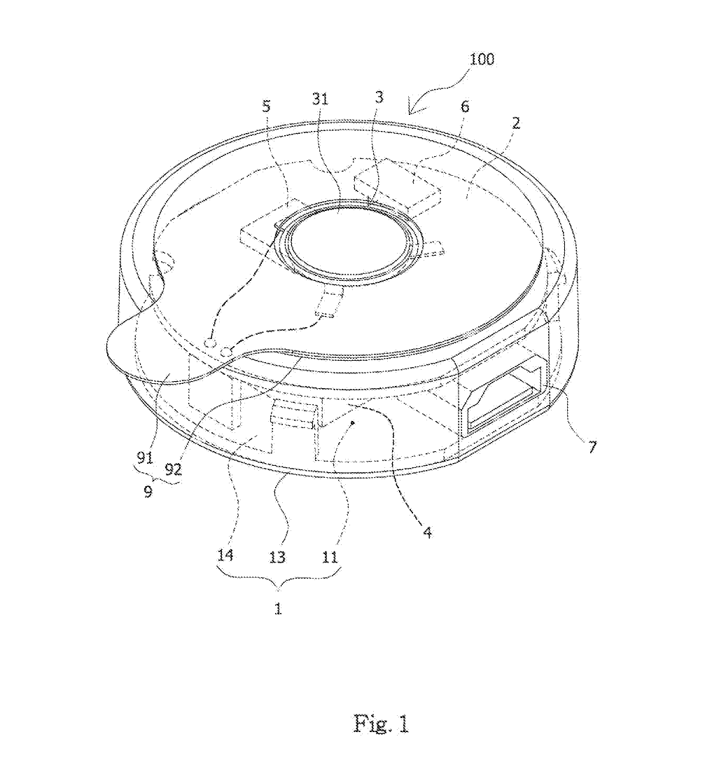

Referring to FIG. 1-4, the Figs. discloses a temperature sensing patch capable of being used with a reading device (A), comprising the temperature sensing patch (100) capable of being attach to human skin, comprising a housing (1), a circuit board (2), a temperature sensing unit (3) electrically connected with the circuit board (2), a battery (4), a processor chip (5), at least a memory unit (6), and at least a port (7); wherein the processor chip (5) can automatically turn on the temperature sensing patch (100) and reset the previous data by a predetermined program, rather than through a manual power switch, when the temperature sensing patch (100) is removed from the reading device (A).

wherein the housing (1) comprises a receiving space (11) defined therein; wherein the circuit board (2) is located within, the received space (11); wherein the temperature sensing unit (3) electrically connected with the circuit board (2) defines a sensing terminal (31) with one end thereof protruded over a surface of the housing (1) to contact with the human skin for sensing temperature; wherein the battery (4) is installed within the receiving space (11); wherein the processor chip (5) is installed on the circuit board (2) and be capable of dealing with the continuous digital data transmitted from the temperature sensing unit (3) within a predetermined time; wherein the memory unit (6) is installed on the circuit board (2) and stored the continuous digital data transmitted from the processor chip (5); and wherein the port (7) is installed within the receiving space (11), one end of which is located at a peripheral edge of the housing (1) for connecting with the reading device.

When in use, the temperature sensing patch (100) can be used by multi-point monitoring method to monitor the body temperature change for a long period time, so the temperature sensing patch (100) is, not as familiar as traditional thermometer does, it can provide the more practical reference data to the precision medicine.

Besides, there is no power switch in the temperature sensing patch (100), so that it can automatically turn on the power through the predetermined program when the temperature sensing patch (100) is removed from the reading device, and then the temperature. sensing patch (100) will turn itself off when the battery runs out of power, so that the design can reduce user's burden on operation.

Moreover, the temperature sensing patch (100) can be synchronously used with multi-point measurement at the same time, thereby avoiding the data error generated by measuring in different time.

Furthermore, except for controlling the switch of the temperature sensing patch (100) and handling digital information transmitted from the temperature sensing unit (3) within a predetermined time period, the processor chip (5) can also secure the data stored in the memory unit (6) before running out of the battery; the temperature sensing patch (100) is capable of transmitting the data and recharging the battery (4) through the port (7).

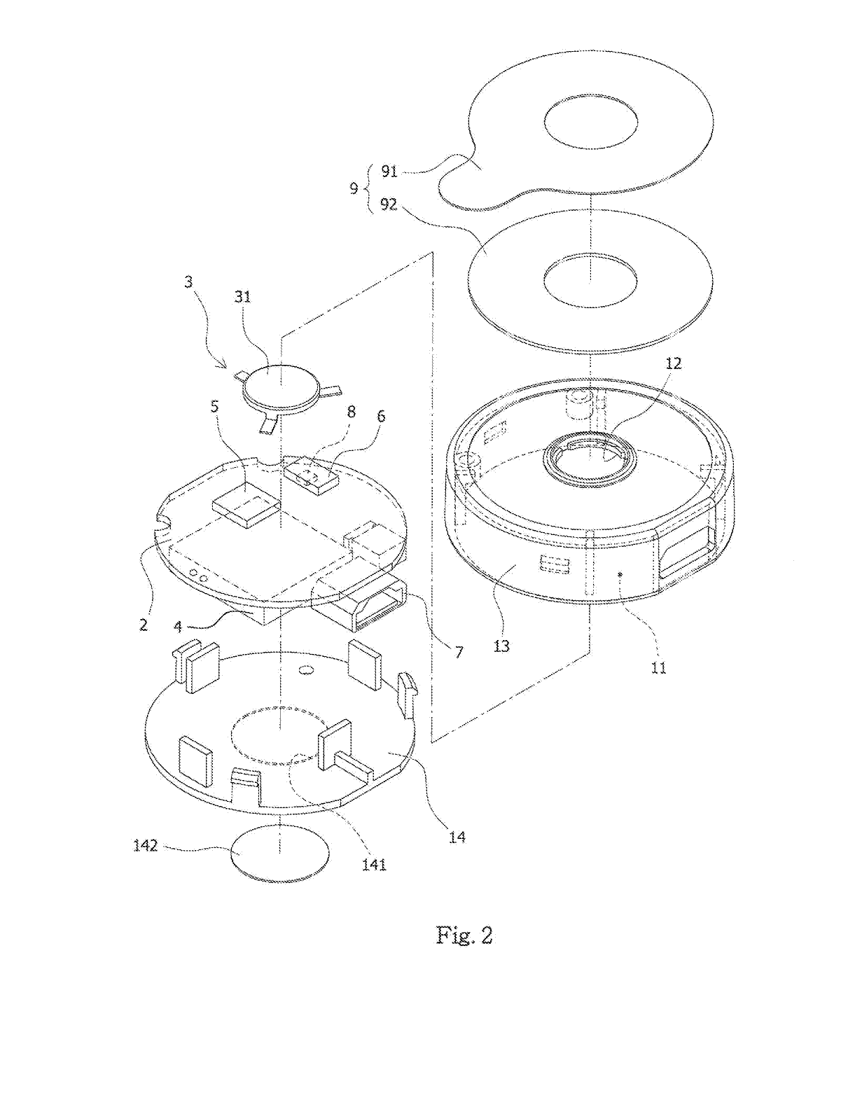

Referring to FIG. 1-2, wherein the temperature sensing patch (100) further comprises a light unit (8) electrically connected with the circuit hoard (2) and located within the received space (11) for a light indication of operation status.

The light unit (8) is configured to identify whether or not the temperature sensing, patch (100) is in use.

Wherein the housing (1) comprises a through hole (12) corresponding to the sensing terminal (31), whereby the through hole (12) is configured to fix the sensing terminal (31).

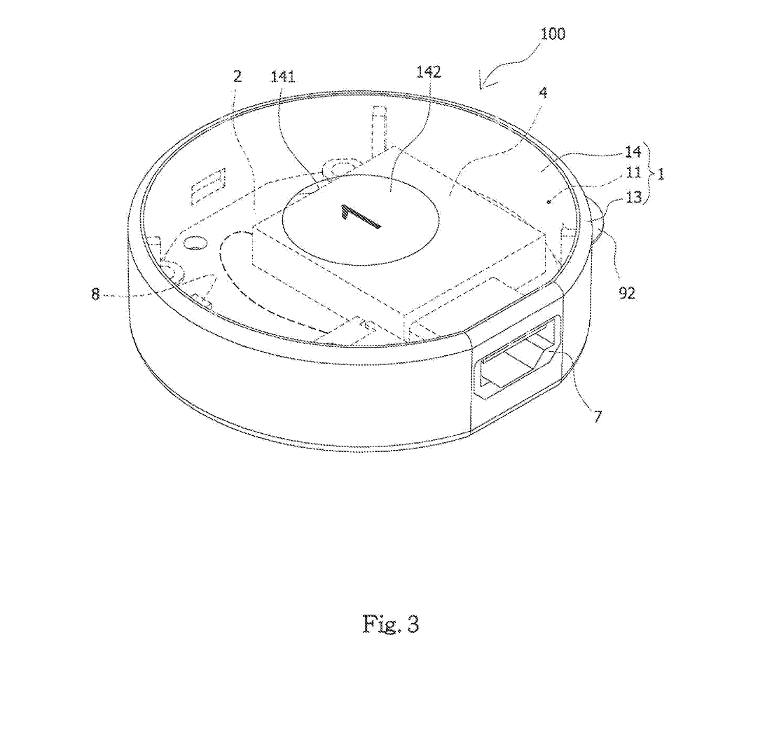

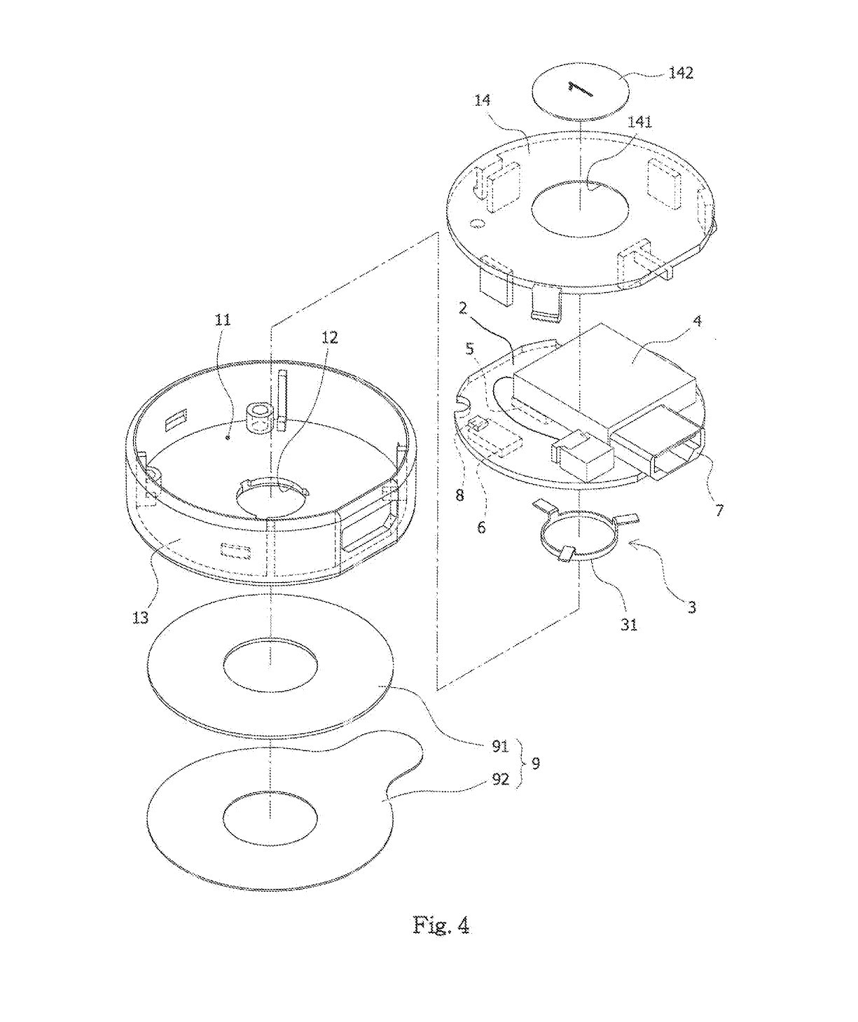

Referring to FIG. 3-4, wherein the temperature sensing patch (100) further comprises an adhesive unit (9) disposed on a surface of the housing (1) and located around a periphery of the through hole (12) for attaching on the human skin. Wherein the adhesive unit (9) comprises an adhesive layer (91) and a release layer (92) laminating with the adhesive layer (91).

The temperature sensing patch (100) can be directly attached on the human's skin after removed from the release layer. Besides, for complying with the medical standards, the adhesive unit (9) can be reusable or disposable.

Wherein the housing (1) further comprises a first housing (13) and a second housing (14) correspondingly disposed on the first housing (13).

The structure of the first and second housing (13, 14) is simple and easy to produce and assemble, and efficient to protect the internal devices.

Wherein the second housing (14) comprises a receiving groove (141) disposed in a side on top thereof and an identification sticker (142) disposed in the receiving groove (141) and configured to identify the temperature sensing patch (2).

In addition to making the identification sticker (142) easy to fix, the receiving groove (141) can avoid the identification sticker (142) accidentally shedding off the second housing (14) caused by wear after using it for a long time.

Wherein the battery (4) can provide the temperature sensing patch (100) with at least 24-hour staying power.

At least the 24-hour staying power of battery capacity for the temperature sensing patch (100) can obtain more temperature data from human body based on the a person's daily activity. Besides, there is no manual power switch installed on the temperature sensing patch (100), so the light unit (8) would automatically light up to identify the battery power when in use, if the battery (4) is running out of power, the light unit (8) will be changed to other color and/or twinkling light to alert users.

Wherein the port (7) is one of the following: Type-A USB port, Type-B USB port, Type-C USB port, Micro-A USB, Micro-B USB, Mini-A USB port, Mini-B USB port. Wherein the light unit (8) is a LED light.

The USB applied in the port (7) not only can transmit the data and recharge the battery at the same time, but also can reduce the size of the temperature sensing patch (100). Besides, the LED light applied in the light unit (8) not only can be easy to determine the operation status of the temperature sensing patch (100), but also can save more power and extend the use life.

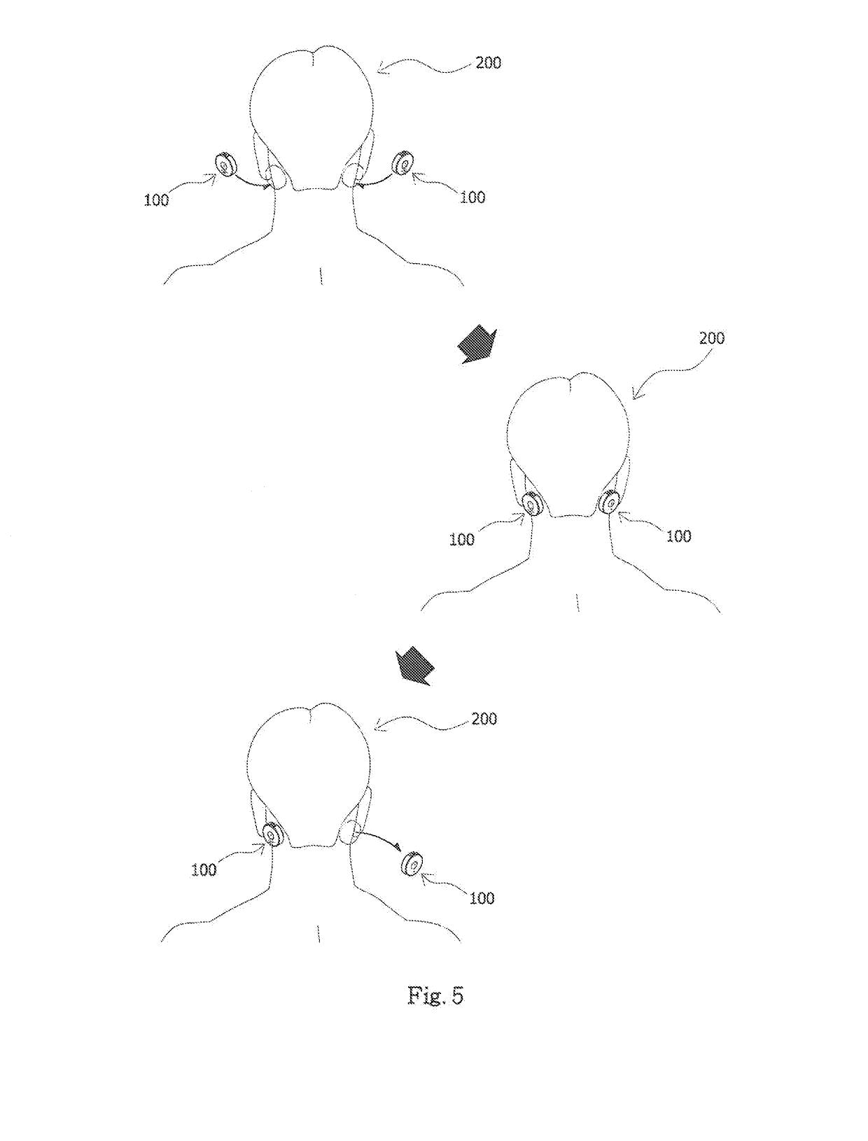

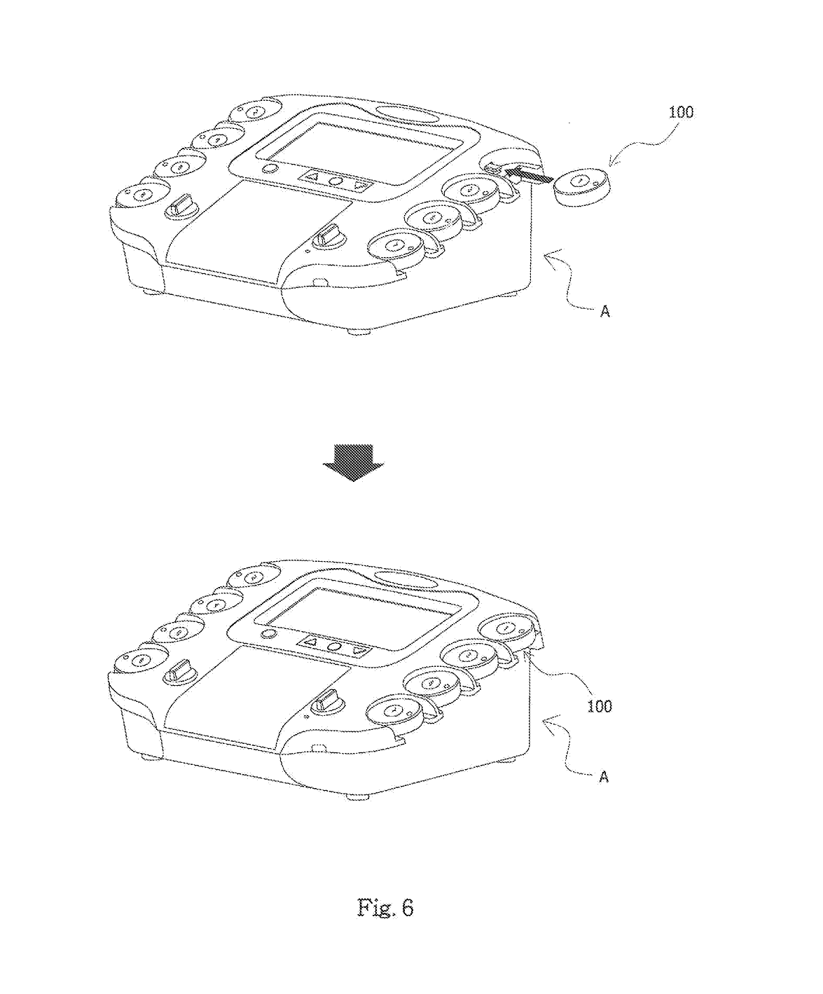

Referring to FIG. 5-6, a using method of the present invention is to put a plurality of the temperature sensing patch (100) on a predetermined position of a participant (200) to make the metal sensing terminal (31) contacting with the human's skin, so that the body temperature data is transmitted to the sensing terminal (31) and the temperature, data is stored in the memory unit (6) through the processor chip (5); wherein the predetermined position is a location of lesion diagnosed by a doctor; after 24 hours of multi-point measurement, the body temperature change data is obtained from the participant (200), and then a curve chart and data of the body temperature change are generated by the temperature sensing patch (100) being plugged into the reading device (A).

the purpose of obtaining the body temperature change data is based on the traditional Chinese medicine theory which holds that the human body is viewed as a whole system that has nature operation and balance, the human will get sick if human body is out of balance, therefore the temperature change represents the balance change, the location of the out-of-balance can be determined by the curve chart and the body temperature change data, thereby facilitating diagnosing the pathology and etiology of the participant (200).

It will be evident to those skilled in the art that the invention is not limited to the details of the foregoing illustrated embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

I claim:

1. A temperature sensing patch capable of being used with a reading device (A), comprising
the temperature sensing patch (100) configured to attach to human skin, comprising a housing (1), a circuit board (2), a temperature sensing unit (3) electrically connected with the circuit board (2), a battery (4), a processor chip (5), at least a memory unit (6), and at least a port (7)
wherein the processor chip (5) is capable of automatically turning on the temperature sensing patch (100) and resetting the previous data by a predetermined program, rather than through a manual switch, when the temperature sensing patch (100) is removed from the reading device (A).

2. The temperature sensing patch according to claim 1, wherein the housing (1) comprises a receiving space (11) defined therein; wherein the circuit board (2) is located within the received space (11); wherein the temperature sensing unit (3) electrically connected with the circuit board (2) defines a sensing terminal (31) with one end thereof protruding over a surface of the housing (1) to contact with the human skin for sensing temperature; wherein the battery (4) is installed within the receiving space (11); wherein the processor chip (5) is installed on the circuit board (2) and is capable of dealing with continuous digital data transmitted from the temperature sensing unit (3) within a predetermined time; wherein the, memory unit (6) is installed on the circuit board (2) and is configured to store the continuous digital data transmitted From the processor chip (5); and wherein the port (7) is installed within the receiving space (11), one end, of which is located at a peripheral edge of the housing (1) for connecting with the reading device

3. The temperature sensing patch according to claim 1, wherein the temperature sensing patch (100) further comprises a light unit (8) electrically connected with the circuit board (2) and located within the received space (11) for a light indication of operation status of the temperature sensing patch (100).

4. The temperature sensing patch according to claim 1, wherein the housing (1) comprises a through hole (1) corresponding to the sensing, terminal (31).

5. The temperature sensing patch according to claim 3, wherein the temperature sensing patch (100) further comprises an adhesive unit (9) disposed on a top surface of one side of the housing (1) and located around a periphery of the through hole (12) for attaching on the human skin.

6. The temperature sensing patch according to claim 5, wherein the adhesive unit (9) comprises an adhesive layer (91) and a release layer (92) laminating with the adhesive layer (91).

7. The temperature sensing patch according to claim 1, wherein the housing (1) further comprises a first housing (13) and a second housing (14) correspondingly disposed on the first housing (13).

8. The temperature sensing patch according to claim 7, wherein the second housing (14) comprises a receiving groove (141) disposed in a side on top thereof and an identification sticker (142) disposed in the receiving groove (141) and configured to identify the temperature sensing patch (2).

9. The temperature sensing patch according to claim 1 wherein the battery (4) is capable of providing the temperature sensing patch (100) with at least 24-hour staying power.

10. The temperature sensing patch according to claim 1, wherein the port (7) is one of the following: Type-A USB port, Type-B USB port, Type-C USB port, Micro-A USB, Micro-B USB, Mini-A USB port, Mini-USB port.

11. The temperature sensing patch according to claim 1, wherein the light unit (8) is a LED light.
